(19) United States
(12) Patent Application Publication
(10) Pub. No.: US 2024/0149007 A1
(43) Pub. Date: May 9, 2024

(54) LIGHT-ASSISTED SLEEP AND WAKE-UP ALARM CLOCK AND A WAKE-UP METHOD THEREOF

(71) Applicant: HAI ZHOU, Shenzhen (CN)

(72) Inventor: HAI ZHOU, Shenzhen (CN)

(21) Appl. No.: 17/980,852

(22) Filed: Nov. 4, 2022

(51) Int. Cl. A61M 21/00; G04G 13/02; G04G 21/02

(52) U.S. Cl. CPC A61M 21/00; G04G 13/026; G04G 21/025; A61M 2021/0027; A61M 2021/0044; A61M 2021/0083; A61M 2205/3303; A61M 2205/3368; A61M 2205/3569; A61M 2210/083

(57) ABSTRACT

The present application provides a light-assisted sleep and wake-up alarm clock and a wake-up method thereof, and relates to the field of human biological sleep. The light sleep aid and wake-up alarm clock comprises an alarm clock and a sleep monitoring device, a light-emitting unit is arranged on the front of the alarm clock, a timer is arranged in the middle of the alarm clock, and a control module is arranged inside the alarm clock and a control mainboard is electrically connected with the light-emitting unit and the timer, a wireless receiving module is also arranged on the control mainboard, a wireless sending module is arranged inside the sleep monitoring device, and the wireless sending module is wirelessly connected with the wireless receiving module, and a sleep aid module and a sleep detection module are also arranged inside the sleep monitoring device.

### TECHNICAL FIELD

The present application relates to the field of human biological sleep, in particular to a light-assisted sleep and wake-up alarm clock and a wake-up method thereof.

### BACKGROUND

Melatonin is a hormone secreted by the pineal gland in the human brain. Scientific research has shown that melatonin is an important factor in regulating our biological clock, and it is this hormone that guides us to fall asleep and improve sleep quality. Therefore, promoting and inhibiting the secretion of melatonin can achieve the purpose of sleep aid and awakening.

There is a small amount of cells in our retina called non-imaging visual cells. As the name suggests, these cells do not help us see, but can perceive light. When these cells sense light, they send a signal to our circadian pacemaker, the suprachiasmatic nucleus, which in turn informs the pineal gland to secrete melatonin, which regulates our biological clock. When the light is bright, the secretion of melatonin is low, and when the light is weakened, the secretion of melatonin increases.

In 2000, Harvard Medical School, Brigham and Women's Hospital and other institutions, through a nine-day experiment on 23 volunteers, concluded that light has a significant effect on the secretion of melatonin in the human body.

A study published in the July 2010 issue of the Chinese Journal of Sports Medicine showed that 30 minutes of red light exposure per day helped women basketball players improve post-training sleep quality.

An existing light wake-up alarm clock method on the market is to simply use light intensity stimulation, that is, slowly increase the brightness of the light to wake people up slowly. This method is relatively simple, but the effect is poor and single.

The research shows that the shorter wavelengths of light at 470, 495 and 525 nm make the secretion of melatonin the most advanced, about 40-60 minutes earlier. This more accurately shows that the effects of different wavelengths of light on the secretion of melatonin in the human body are significantly different. Irradiating the retina with red light can promote the secretion of melatonin, so that the human body can quickly enter a sleep state, and irradiating the retina with green light can inhibit the secretion of melatonin and make the brain wake up quickly, thereby regulating the rhythm of the human body's biological clock. Based on the above principle, the present application develops a light-assisted sleep and wake-up alarm clock and a wake-up method thereof.

### SUMMARY

In view of the deficiencies of the prior art, the present application provides a light-assisted sleep and wake-up alarm clock and a wake-up method thereof, which can release light of different wavelengths to promote or inhibit the secretion of melatonin based on the law of the human body biological clock, thereby improving sleep state.

To achieve the above purpose, the present application is achieved through the following technical solutions: A light-assisted sleep and wake-up alarm clock, comprising an alarm clock and a sleep monitoring device, wherein a light-emitting unit is arranged on front of the alarm clock, a timer is arranged in middle of the alarm clock, and a control module is arranged inside the alarm clock and a control mainboard is electrically connected with the light-emitting unit and the time, a wireless receiving module is also arranged on the control mainboard, a wireless sending module is arranged inside the sleep monitoring device, and the wireless sending module is wirelessly connected with the wireless receiving module, and a sleep aid module and a sleep detection module are also arranged inside the sleep monitoring device.

Preferably, the sleep aid module comprises a sleep mode module and a wake-up mode module.

Preferably, the sleep monitoring module comprises a body temperature monitoring sensor, a pulse sensor, a capacitive skin contact sensor and an EEG signal acquisition unit, and the capacitive skin contact sensor comprises four metal conductors and a sensor controller.

Preferably, a player is arranged inside the timer, and the player is configured to play white noise for assisting sleep.

A wake-up method by using the light-assisted sleep and wake-up alarm clock, comprising following steps:

Step S1: put the sleep monitoring device on a wrist, and adjust the sleep monitoring device to enter the sleep mode module:

Step S2: send a monitoring information to the wireless receiving module by the wireless sending module, and emit red light by the light-emitting unit under control of the control board, to promote the secretion of melatonin in a human body, and makes the human body quickly enter a sleep state;

Step S3: determine whether to enter the sleep state through the sleep monitoring module, if so, execute step S4, otherwise return to step S2:

Step S4: turn off the light-emitting unit by the control mainboard, and at the same time make the timer enter a timing mode;

Step S5: end the timing mode, and make the sleep monitoring device enter the wake-up mode module:

Step S6: send the monitoring information to the wireless receiving module by the wireless sending module, and emit blue-green light through the light-emitting unit under control of the control mainboard, to inhibit the secretion of melatonin in the human body and makes the brain wake up quickly;

Step S7: determine whether to enter a wake-up state through the sleep monitoring module, if so, execute step S8, otherwise return to step S6:

Step S8: End, turn off the light-emitting unit under control of the control mainboard.

The present application provides a light-assisted sleep and wake-up alarm clock and a wake-up method thereof, which has the following beneficial effects:

According the present application, the control mainboard controls the light-emitting unit to emit lights of different wavelengths and colors in different sleep states to promote or inhibit the secretion of melatonin in the human body, thereby helping the human body to quickly sleep and wake up and improve sleep quality. The structure is simple, the operation is convenient, and is healthy and effective.

### BRIEF DESCRIPTION OF DRAWINGS

FIG. 1 is the schematic diagram of the alarm clock in the present application;

FIG. 2 is a schematic diagram of a sleep monitoring device in the present application;

FIG. 3 is a schematic diagram of the system working flow of the present application.

### REFERENCE SIGNS

1. Alarm clock; 2. Sleep monitoring device; 3. Light-emitting unit; 4. Timer.

### DETAILED DESCRIPTION

The technical solutions in the embodiments of the present application will be clearly and completely described below with reference to the accompanying drawings in the embodiments of the present application. Obviously, the described embodiments are only a part of the embodiments of the present application, but not all of the embodiments. Based on the embodiments of the present application, all other embodiments obtained by those of ordinary skill in the art without creative efforts shall fall within the protection scope of the present application.

### Embodiment

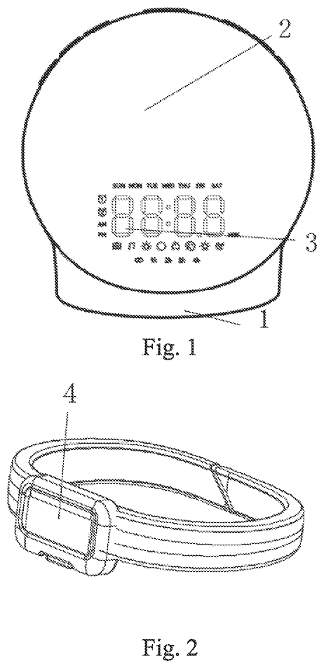

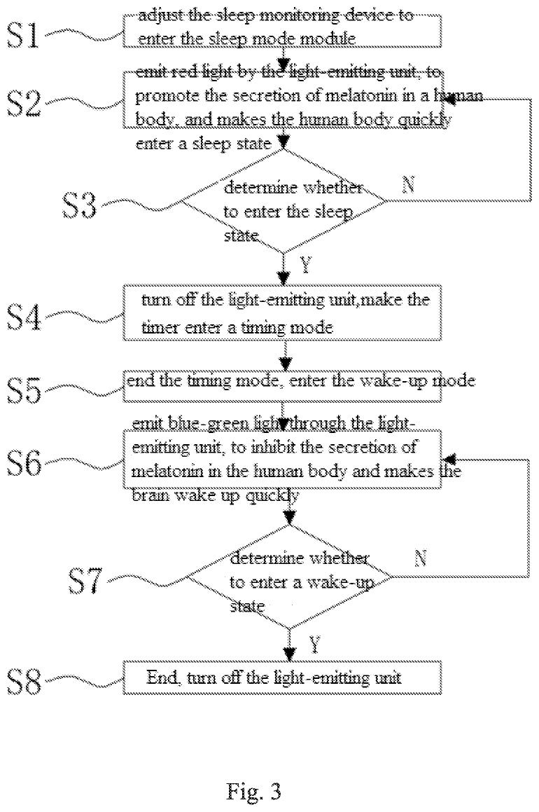

As shown in FIGS. 1-3, an embodiment of the present application provides a light-assisted sleep and wake-up alarm clock, which comprises an alarm clock 1 and a sleep monitoring device 2, wherein a light-emitting unit 3 is arranged on front of the alarm clock 1, such as light-emitting diodes, neon lights, fluorescent lights or RGB light strips, can be used as light-emitting units, which support multiple color outputs to assist sleep. A timer 4 is arranged in middle of the alarm clock 1, which is used for timing and displaying time, wherein a player is arranged inside the timer 4, and the player is configured to play white noise for assisting sleep. A control module is arranged inside the alarm clock 1 and a control mainboard is electrically connected with the light-emitting unit 3 and the timer 4, a wireless receiving module is also arranged on the control mainboard, a wireless sending module is arranged inside the sleep monitoring device 2, and the wireless sending module is wirelessly connected with the wireless receiving module, and a sleep aid module and a sleep detection module are also arranged inside the sleep monitoring device 2.

Specifically, turn on the sleep mode when you are going to sleep at night, and the light-emitting unit 3 will emit a special red-orange light of 590-750 nanometers, which promotes the secretion of melatonin and accelerates the onset of sleepiness. In the morning, the special red-orange light with a wavelength of 750 nanometers slowly changes to a special blue-green light with a wavelength of 470 nanometers at a set time. This light change can slowly inhibit the melatonin in the body, allowing people to wake up healthier and naturally, and make people more spirited during the day.

Melatonin is a hormone secreted by the pineal gland in the human brain, and it is this hormone that helps us fall asleep and improve sleep quality.

Furthermore, the sleep aid module includes a sleep mode module and a wake-up mode module, which are used to perform program operations during sleep aid and wake-up, respectively.

Furthermore, the sleep monitoring module comprises a body temperature monitoring sensor, a pulse sensor, a capacitive skin contact sensor and an EEG signal acquisition unit, and the capacitive skin contact sensor comprises four metal conductors and a sensor controller. The sensor controller detects the capacitance information generated between the metal conductors in real time. The pulse sensor is a reflective photoelectric pulse sensor, which is used to detect the human heart rate in real time. The Body temperature monitoring sensor is used to monitor body temperature during sleep. The electroencephalogram (EEG) signal acquisition unit is used to collect the electroencephalogram signals generated during sleep. Combine all the collected data to determine the specific state of sleep.

A wake-up method by using the light-assisted sleep and wake-up alarm clock, comprising following steps:

Step S1: put the sleep monitoring device on a wrist, and adjust the sleep monitoring device to enter the sleep mode module:

Specifically, the sleep monitoring device is in the form of a bracelet, which can be bound on a wrist through a strap. The bracelet is provided with a control screen and buttons, and the bracelet can be adjusted through the control screen to enter the sleep mode module, which can not only monitor the users sleep mode in real time, but also not interfere with the normal sleep state of the user.

Step S2: send a monitoring information to the wireless receiving module by the wireless sending module, and emit red light by the light-emitting unit under control of the control board, to promote the secretion of melatonin in a human body, and makes the human body quickly enter a sleep state;

Specifically, the alarm clock needs to be placed on the bedside or close to the eyes of the sleeper. When the light-emitting unit emits red light, the users eyes can receive the red light information, thereby smoothly promoting human melatonin. The secretion is convenient for the user to quickly enter the sleep state.

Step S3: determine whether to enter the sleep state through the sleep monitoring module, if so, execute step S4, otherwise return to step S2;

Specifically, through the body temperature monitoring sensor, the pulse sensor, the capacitive skin contact sensor and the EEG signal acquisition unit on the sleep detection module, the body temperature monitoring, the human heart rate monitoring, the generated capacitive signal monitoring and the brain wave signal monitoring are performed on the user during sleep, so as to accurately analyze the users sleep state.

Step S4: turn off the light-emitting unit by the control mainboard, and at the same time make the timer enter a timing mode;

Specifically, the timer 3 can manually set the sleep duration according to the users needs, and the timer mode is turned on or off according to the state of the light-emitting unit 2, which is convenient for the user to grasp the real sleep duration.

In another embodiment, if the user can enter deep sleep even in a lighted environment, the light-emitting unit 2 can be kept on, so that the human body can continuously secrete melatonin and improve sleep quality. When the user cannot enter the deep sleep normally in a lighted environment, the light emitting unit 2 can be turned off after the user enters the light sleep to prevent interference to the user.

Step S5: end the timing mode, and make the sleep monitoring device enter the wake-up mode module:

Step S6: send the monitoring information to the wireless receiving module by the wireless sending module, and emit blue-green light 3 through the light-emitting unit under control of the control mainboard, to inhibit the secretion of melatonin in the human body and makes the brain wake up quickly;

Specifically, the wake-up mode module is wirelessly connected to the timer 3, and when the timing mode of the timer 3 ends, the sleep mode module automatically transfers to the wake-up mode module.

Step ST determine whether to enter a wake-up state through the sleep monitoring module, if so, execute step S8, otherwise return to step S6;

Specifically, if it is determined that the user is in the light sleep mode through the body temperature monitoring sensor, the pulse sensor, the capacitive skin contact sensor, and the EEG signal acquisition unit in the sleep monitoring module, the light-emitting unit 3 can be controlled to alternately change blue and green light or to change the integrated color slowly, so as to provide a more comfortable wake-up state and help the brain wake up quickly.

Step S8: End, turn off the light-emitting unit under control of the control mainboard.

Adjusting the body's biological clock through light with different wavelengths can not only wake up more healthily and efficiently, but also assist sleep and improve the rhythm of the human body's biological clock.

Although embodiments of the present application have been shown and described, it will be understood by those skilled in the art that various changes, modifications, and substitutions can be made in these embodiments without departing from the principle and spirit of the application and modifications, the scope of the present application is defined by the appended claims and their equivalents.

What is claimed is:

1. A light-assisted sleep and wake-up alarm clock, comprising an alarm clock (1) and a sleep monitoring device (2), wherein a light-emitting unit (3) is arranged on front of the alarm clock (1), a timer (4) is arranged in middle of the alarm clock (1), and a control module is arranged inside the alarm clock (1) and a control mainboard is electrically connected with the light-emitting unit (3) and the timer (4), a wireless receiving module is also arranged on the control mainboard, a wireless sending module is arranged inside the sleep monitoring device (2), and the wireless sending module is wirelessly connected with the wireless receiving module, and a sleep aid module and a sleep detection module are also arranged inside the sleep monitoring device (2).

2. The light-assisted sleep and wake-up alarm clock according to claim 1, wherein the sleep aid module comprises a sleep mode module and a wake-up mode module.

3. The light-assisted sleep and wake-up alarm clock according to claim 1, wherein the sleep monitoring module comprises a body temperature monitoring sensor, a pulse sensor, a capacitive skin contact sensor and an EEG signal acquisition unit, and the capacitive skin contact sensor comprises four metal conductors and a sensor controller.

4. The light-assisted sleep and wake-up alarm clock according to claim 1, wherein a player is arranged inside the timer (4), and the player is configured to play white noise for assisting sleep.

5. A wake-up method by using the light-assisted sleep and wake-up alarm clock according to claim 1, comprising following steps:
Step S1: put the sleep monitoring device (2) on a wrist, and adjust the sleep monitoring device (2) to enter the sleep mode module;
Step S2: send a monitoring information to the wireless receiving module by the wireless sending module, and emit red light by the light-emitting unit (3) under control of the control board, to promote the secretion of melatonin in a human body, and makes the human body quickly enter a sleep state;
Step S3: determine whether to enter the sleep state through the sleep monitoring module, if so, execute step S4, otherwise return to step S2;
Step S4: turn off the light-emitting unit (3) by the control mainboard, and at the same time make the timer (4) enter a timing mode;
Step S5: end the timing mode, and make the sleep monitoring device (2) enter the wake-up mode module;
Step S6: send the monitoring information to the wireless receiving module by the wireless sending module, and emit blue-green light through the light-emitting unit (3) under control of the control mainboard, to inhibit the secretion of melatonin in the human body and makes the brain wake up quickly;
Step S7: determine whether to enter a wake-up state through the sleep monitoring module, if so, execute step S8, otherwise return to step S6;
Step S8: End, turn off the light-emitting unit under control of the control mainboard.
